(19) United States
(12) Patent Application Publication
(10) Pub. No.: US 2024/0307446 A1
(43) Pub. Date: Sep. 19, 2024

(54) Generation and Utility of B Cell Subsets for Treatment of Chronic Obstructive Pulmonary Disease

(71) Applicant: Therapeutic Solutions International, Inc., OCEANSIDE, CA (US)

(72) Inventors: THOMAS E. ICHIM, OCEANSIDE, CA (US); TIMOTHY G. DIXON, OCEANSIDE, CA (US); JAMES VELTMEYER, OCEANSIDE, CA (US)

(73) Assignee: Therapeutic Solutions International, Inc., OCEANSIDE, CA (US)

(21) Appl. No.: 18/605,746

(22) Filed: Mar. 14, 2024

Related U.S. Application Data
(60) Provisional application No. 63/452,101, filed on Mar. 14, 2023.

(51) Int. Cl. A61K 35/17; A61K 39/00; A61P 11/00; C07K 14/475; C07K 14/54; C07K 14/705; C07K 14/715; C12N 5/071; C12N 5/0783

(52) U.S. Cl. CPC A61K 35/17; A61K 39/4612; A61K 39/46441; A61K 39/464419; A61K 39/464429; A61K 39/46444; A61P 11/00; C07K 14/4753; C07K 14/5428; C07K 14/70596; C07K 14/7155; C12N 5/0646; C12N 5/0688

(57) ABSTRACT

B cell subsets, generation of B cell subsets and utilization of B cell subsets for treatment of Chronic Obstructive Pulmonary Disease (COPD). In one embodiment B cells possessing a B regulatory phenotype are generated in vivo by administrating of mesenchymal stem cells. In another embodiment B regulatory cells are utilized to treat COPD in an interleukin-35 dependent manner. In another embodiment B regulatory cells possess the marker CD5 and produce interleukin-10.

### CROSS-REFERENCE TO RELATED APPLICATIONS

The present application claims benefit of U.S. Provisional Patent Application Ser. No. 63/452,101, filed on Mar. 14, 2023, entitled GENERATION AND UTILITY OF B CELL SUBSETS FOR TREATMENT OF CHRONIC OBSTRUCTIVE PULMONARY DISEASE, the contents of which are incorporated herein by reference in its entirety.

### FIELD OF THE INVENTION

The teachings herein related to the treatment of COVID using B cell subsets.

### BACKGROUND OF THE INVENTION

At least one specification heading is required. Please delete this heading section if it is not applicable to your application. For more information regarding the headings of the specification, please see MPEP 608.01(a).

COPD stands for Chronic Obstructive Pulmonary Disease, which is a progressive lung disease that makes it difficult for a person to breathe. The disease is typically characterized by chronic bronchitis and emphysema, which are both caused by long-term exposure to irritating gases or particulate matter, such as cigarette smoke or air pollution. Chronic bronchitis is a condition where the bronchial tubes become inflamed and produce excess mucus, causing a persistent cough and difficulty breathing. Emphysema, on the other hand, is a condition where the air sacs in the lungs are damaged and lose their elasticity, making it difficult for the lungs to empty air. COPD is a leading cause of death worldwide, and it is estimated that more than 16 million people in the United States alone are diagnosed with COPD. Symptoms of COPD include shortness of breath, wheezing, chest tightness, and a chronic cough. While there is no cure for COPD, treatments such as medication, oxygen therapy, and pulmonary rehabilitation can help manage symptoms and improve quality of life.

There are several molecular pathways involved in COPD, these include: a) Cytokines. These are small proteins produced by various cells, including immune cells, that regulate inflammation in the lungs. In COPD, cytokines such as IL-1, IL-6, IL-8, and TNF-alpha are elevated, leading to chronic inflammation in the airways; b) Reactive oxygen species (ROS). These are highly reactive molecules produced by cells during oxidative stress. In COPD, ROS are generated by activated immune cells and in response to cigarette smoke exposure, leading to damage to lung tissue and inflammation c) Matrix metalloproteinases (MMPs). These are enzymes that degrade extracellular matrix proteins and are involved in tissue remodeling. In COPD, MMPs are upregulated, leading to destruction of lung tissue and loss of lung function; d) Neutrophils. These are a type of white blood cell that plays a key role in the inflammatory response. In COPD, neutrophils are recruited to the lungs and release enzymes and cytokines that contribute to tissue damage and inflammation; e) Macrophages. These are another type of immune cell that plays a role in the inflammatory response. In COPD, macrophages release cytokines and chemokines that attract other immune cells to the lungs, leading to chronic inflammation and f) Proteases: These are enzymes that break down proteins. In COPD, proteases such as elastase and cathepsin G are released by activated immune cells, leading to destruction of lung tissue and loss of lung function.

Treatments of COPD are not curative, but they include: Medications such as bronchodilators, corticosteroids, and combination inhalers. These medications work to reduce inflammation, relax the airways, and help the patient breathe more easily. Another approach is making certain lifestyle changes can help manage COPD symptoms. These include quitting smoking, avoiding secondhand smoke and other pollutants, and staying physically active. A healthy diet and maintaining a healthy weight can also help. Additionally, pulmonary rehabilitation is a program that combines exercise, breathing techniques, and education to help improve lung function and overall quality of life for people with COPD. It can include things like supervised exercise sessions, nutritional counseling, and support groups. Oxygen therapy is sometimes used for people with severe COPD who have low levels of oxygen in their blood. This involves using oxygen from a tank or concentrator to help improve breathing. Surgery may be an option for some people with severe COPD. Lung volume reduction surgery removes damaged lung tissue, while lung transplant surgery replaces a diseased lung with a healthy one.

### SUMMARY OF THE INVENTION

Preferred embodiments include methods of treating COPD by administration and/or generation of a therapeutic population of B cells.

Preferred methods include embodiments wherein said COPD is associated with excessive production of elastase.

Preferred methods include embodiments wherein said elastase is produced as a result of neutrophil activation.

Preferred methods include embodiments wherein said therapeutic B cells are B regulatory cells.

Preferred methods include embodiments wherein said B regulatory cells express interleukin-10.

Preferred methods include embodiments wherein said B regulatory cells express c-met.

Preferred methods include embodiments wherein said B regulatory cells express thrombopoietin receptor.

Preferred methods include embodiments wherein said B regulatory cells express c-kit.

Preferred methods include embodiments wherein said B regulatory cells express CD5.

Preferred methods include embodiments wherein said B regulatory cells express complement receptor 3.

Preferred methods include embodiments wherein said B regulatory cells express interleukin-7 receptor.

Preferred methods include embodiments wherein said B regulatory cells produce interleukin-35.

Preferred methods include embodiments wherein said B regulatory cells are capable of inducing proliferation of pulmonary type 2 epithelial cells.

Preferred methods include embodiments wherein said B regulatory cells, upon crosslinking of CD19 are capable of modifying nkt cells to produce a reduced amount of interleukin-17 as compared to control nkt cells.

Preferred methods include embodiments wherein said B regulatory cells, upon crosslinking of CD19 are capable of modifying nkt cells to produce a reduced amount of TNF-alpha as compared to control nkt cells.

Preferred methods include embodiments wherein said B regulatory cells, upon crosslinking of CD19 are capable of modifying nkt cells to produce a reduced amount of interferon gamma as compared to control nkt cells.

Preferred methods include embodiments wherein said B regulatory cells, upon crosslinking of CD19 are capable of modifying nkt cells to produce a reduced amount of HMGB1 as compared to control nkt cells.

Preferred methods include embodiments wherein said B regulatory cells, upon crosslinking of CD19 are capable of modifying nkt cells to produce an enhanced amount of interleukin-10 as compared to control nkt cells.

Preferred methods include embodiments wherein said B regulatory cells, upon crosslinking of CD19 are capable of modifying nkt cells to produce an enhanced amount of soluble TNF-alpha receptor p55 as compared to control nkt cells.

Preferred methods include embodiments wherein said B regulatory cells, upon crosslinking of CD19 are capable of modifying nkt cells to produce an enhanced amount of soluble TNF-alpha receptor p75 as compared to control nkt cells.

Preferred methods include embodiments wherein said B regulatory cells, upon crosslinking of CD19 are capable of modifying nkt cells to produce an enhanced amount of interleukin-1 receptor antagonist as compared to control nkt cells.

Preferred methods include embodiments wherein said B regulatory cells, upon crosslinking of CD19 are capable of modifying nkt cells to produce an enhanced amount of soluble HLA-G as compared to control nkt cells.

Preferred methods include embodiments wherein said B regulatory cells, upon crosslinking of CD19 are capable of modifying nkt cells to produce an enhanced amount of GDF-11 as compared to control nkt cells.

Preferred methods include embodiments wherein said B regulatory cells, upon crosslinking of CD19 are capable of modifying nkt cells to produce an enhanced amount of immune modulatory exosomes as compared to control nkt cells.

Preferred methods include embodiments wherein said B regulatory cells are generated in vivo or in vitro by contact with mesenchymal stem cells.

Preferred methods include embodiments wherein said mesenchymal stem cells are pretreated with TNF-alpha.

Preferred methods include embodiments wherein said mesenchymal stem cells are derived from perinatal tissue.

Preferred methods include embodiments wherein said perinatal tissue is umbilical cord plasma.

Preferred methods include embodiments wherein said perinatal tissue is umbilical cord blood.

Preferred methods include embodiments wherein said perinatal tissue is blood derived from a pregnant volunteer.

Preferred methods include embodiments wherein said perinatal tissue is circulating mesenchymal stem cells derived from a pregnant volunteer.

Preferred methods include embodiments wherein said perinatal tissue is umbilical cord matrix.

Preferred methods include embodiments wherein said perinatal tissue is Wharton's Jelly.

Preferred methods include embodiments wherein said perinatal tissue is placenta.

The method of claim 34, wherein said placenta is the chorionic layer of the placenta.

Preferred methods include embodiments wherein said perinatal tissue is amniotic membrane.

Preferred methods include embodiments wherein said perinatal tissue is amniotic fluid.

Preferred methods include embodiments wherein said perinatal tissue is bone marrow from a pregnant volunteer

Preferred methods include embodiments wherein said bone marrow is selected for expression of CD34.

Preferred methods include embodiments wherein said mesenchymal stem cells produce interleukin 1 receptor antagonist upon treatment with lipopolysaccharide.

Preferred methods include embodiments wherein said mesenchymal stem cells are plastic adherent.

Preferred methods include embodiments wherein said mesenchymal stem cells display a fibroblastoid-like morphology.

Preferred methods include embodiments wherein said mesenchymal stem cells are capable of proliferating at a doubling rate of 8-32 hours.

Preferred methods include embodiments wherein said mesenchymal stem cells express CD105.

Preferred methods include embodiments wherein said mesenchymal stem cells express CD73.

Preferred methods include embodiments wherein said mesenchymal stem cells express c-MET.

Preferred methods include embodiments wherein said mesenchymal stem cells possess HIF-alpha that has undergone nuclear translocation.

Preferred methods include embodiments wherein said mesenchymal stem cells express CD37.

Preferred methods include embodiments wherein said mesenchymal stem cells express CD105.

Preferred methods include embodiments wherein said mesenchymal stem cells express c-kit.

Preferred methods include embodiments wherein said mesenchymal stem cells express TNF-alpha receptor p55.

Preferred methods include embodiments wherein said mesenchymal stem cells express TNF-alpha receptor p75.

Preferred methods include embodiments wherein said mesenchymal stem cells express EGF-receptor.

Preferred methods include embodiments wherein said mesenchymal stem cells express NGF-receptor.

Preferred methods include embodiments wherein said mesenchymal stem cells express BDNF-receptor.

Preferred methods include embodiments wherein said mesenchymal stem cells express HLA-G.

Preferred methods include embodiments wherein said mesenchymal stem cells do not express HLA II.

Preferred methods include embodiments wherein said mesenchymal stem cells do not express HLA I.

Preferred methods include embodiments wherein said mesenchymal stem cells do not express CD34.

### BRIEF DESCRIPTION OF DRAWINGS

FIG. 1 is a bar graph showing neutrophil levels in mice after being administered different amounts of B regulatory cell isolated by CD5 expression from mice treated with 1 million MSC.

### DETAILED DESCRIPTION OF THE INVENTION

At least one specification heading is required. Please delete this heading section if it is not applicable to your application. For more information regarding the headings of the specification, please see MPEP 608.01(a).

The invention teaches administration of B regulatory cells as a treatment for COPD. In some embodiments said B regulatory cells are generated in vitro or in vivo using umbilical cord mesenchymal stem cells for treatment of COPD. In some cases administration of said mesenchymal stem cells is performed using cells that have been activated prior to administration. The process of activating said placentally derived mesenchymal is performed in order to augment regenerative, and/or anti-inflammatory, and/or migratory, and/or anti-apoptotic, and/or anti-fibrotic activities of said mesenchymal stem cells.

Isolated regulatory B cells (Breg), and isolated populations of regulatory B cells are disclosed herein. These regulatory B cells are mammalian B cells that express T cell immunoglobulin mucin (TIM)-1 (TIM-1+). In some embodiments, the cells also express CD19, and are TIM-1+CD19+. In additional embodiments, these regulatory B cells also can express CD1d and CD5 (CD1dhighCD5+). In additional embodiments, the regulatory B cells express no or low levels of CD1d or do not express CD5. In some embodiments, the cells are primate cells, such as human or non-human primate cells. In some embodiments, the regulatory B cells express interleukin-10. The ability of the cells to produce IL10 can be assessed by measuring IL-10 production in naive cells and in cultured cells stimulated with LPS (lipopolysaccharide), PMA (phorbol 12-myristate 13-acetate), ionomycin, CpG or comparable stimulatory Toll-like receptor agonists, or with an agonist of CD40 (e.g., using an antibody to CD40). Production of IL-10 by the cells can be assessed by assaying for IL-10 in the cell culture supernatant. In addition, production of IL 10 can be verified directly by intracellular cytokine staining or by Enzyme-linked immunosorbent spot (ELISPOT). Standard immunoassays known in the art can be used for such purpose (see PCT Publication No. 20091131712, which is incorporated herein by reference. An isolated regulatory B cell population can include at least 90%, at least 91%, at least 92%, at least 93%, at least 94%, at least 95% at least 99%, or 100% regulatory B cells that express TIM-1. An isolated regulatory B cell population can include at least 90%, at least 91%, at least 92%, at least 93%, at least 94%, at least 95%, at least 99%, or 100% regulatory B cells that are TIM-1+B cells, such as TIM-1+CD19+B cells that produce IL-10. In some embodiments, a subset of these cells can be CD1dhighCD5+. In some embodiments, the TIM-1+B cells produce IL-10. A regulatory B cell population can include at least 90%, at least 91%, at least 92%, at least 93%, at least 94%, at least 95%, at least 99%, or 100% regulatory B cells that express TIM-1, wherein at least 5% of the TIM-1+B cells produce IL-10. In some embodiments, at least 10%, at least 20%, at least 10%, at least 40%, at least 50% or at least 60% of the TIM-1+B cells in the population produce IL-10. In other embodiments, a regulatory B cell population can include at least 90%, at least 91%, at least 92%, at least 93%, at least 94%, at least 95%, at least 99%, or 100% regulatory B cells that express TIM-1, and express IL-10. Additional isolated populations of cells are disclosed herein. In some embodiments, a regulatory B cell population can include at least 90%, at least 91%, at least 92%, at least 91%, al least 94%, at least 95%, al least 99%, or 100% regulatory B cells that express TIM-1, and are CD1dhighCD5+. In other embodiments, a regulatory B cell population can include at least 90%, at least 91%, at least 92%, at least 93%, at least 94%, at least 95%, at least 99%, or 100% regulatory B cells that express TIM-1, and are CD1dlow/ICD5−. In some embodiments, the disclosed regulatory B cells suppress resting and activated T cells. These regulatory B cells can induce generation of regulatory T cells (Tregs) from CD4+ T cells, for example CD4+FoxP3+ T cells, or can enhance the activity these T cells. In other embodiments, regulatory B cells may suppress other B cells, dendritic cells or macrophages. In specific non-limiting examples, the cells are human, non-human primate or murine cells. TIM-1+ regulatory B cells, such as TIM-1+CD19+B cells prevent the activation and/or expansion of other cells of the immune system. Therefore, in one embodiment, the TIM-1+CD19+ cells are immunosuppressive cells. In additional embodiments, the TIM-1+CD19+ cells are regulatory and produce IL-10. The enriched, isolated and/or purified TIM-1+ regulatory B cells can be obtained from a mammalian subject, including but not limited to rodents, e.g. mice, rats; livestock, e.g. pigs, horses, cows, etc., pets, e.g. dogs, cats; and primates, e.g. humans. In one embodiment, the cells are human TIM-1+ regulatory B culls, for example, TIM-1+CD19+ regulatory B cells. Methods are also provided herein for generating regulatory B cells. These methods include contacting a sample comprising B cells in vitro with an antibody that specifically induces TIM-1, and isolating TIM-1+B cells, such as TIM-1+CD19+B cells. Exemplary non-limiting methods are provided in the Examples section below. The population of regulatory B cells can be obtained from a subject in need of therapy or suffering from a disease associated with reduced regulatory B cell activity. Thus, the cells will be autologous to the subject in need of therapy. Alternatively, the population of regulatory B cells can be obtained from a donor, preferably a histocompatibility matched donor. The regulatory B cell population can be harvested from the peripheral blood, bone marrow, spleen, or any other organ/tissue in which regulatory B cells reside in said subject or donor. The regulatory B cells can be isolated from a pool of subjects and/or donors, or from pooled blood.

The invention teaches use of mesenchymal stem cell, in one particular embodiment, mesenchymal stem cell possessing initially CD34 and CD73, for treatment of COPD. In one particular embodiment, COPD is caused in party by cytokine upregulation, as well as subacute production of disseminated intravascular coagulation response causing degeneration of the alveoli. In one specific embodiment, mesenchymal stem cell are primed with an inflammatory or proinflammatory signal, in order to elicit a corresponding anti-inflammatory and pro-regenerative profile. Within the context of the invention is the novel finding that prestimulation of mesenchymal stem cell with activated protein C (APC) is disclosed as a means of increasing anti-inflammatory potency of mesenchymal stem cell. Said anti-inflammatory potency may be utilized as a means of protecting animals or patients from COPD and inducing regeneration of pulmonary tissue. The invention teaches means of selecting mesenchymal stem cell for enhanced efficacy based on expression of CD73, or lack of expression of certain proteins. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using mesenchymal stem cell-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent. As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotic s/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium. Also relating to the present invention, the term standard growth conditions, as used herein refers to culturing of cells at 37° C., in a standard atmosphere comprising 5% CO2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO2, relative humidity, oxygen, growth medium, and the like. The use of mesenchymal stem cell for treatment of COPD may be based, in one embodiment of the invention, on the reduction of pathological immunological parameters associated with COPD.

### Example

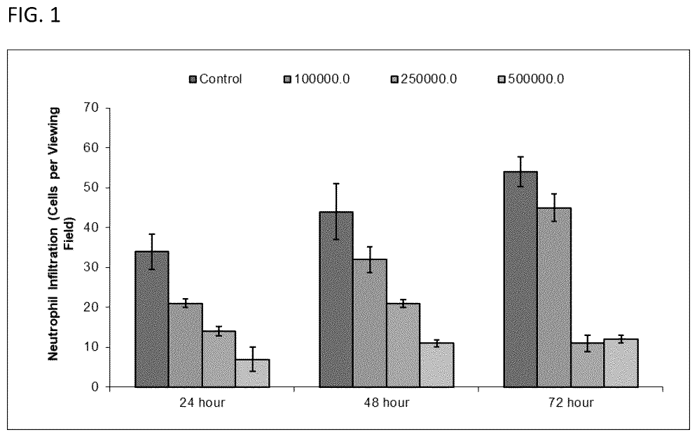

Male 7-week-old C57BL/6 mice were housed in the laboratory animal center under 22±2° C., 55%+10% humidity, and 12 h dark/light cycle conditions. All of the mice were randomized before the experiment. The model was established by intratracheal (IT) instillation of 0.2 IU porcine pancreatic elastase (Sigma-Aldrich, St. Louis, MO, USA) using a microsprayer aerosolizer (Penn-Century, Wyndmoor, PA, USA) under general anesthesia with 3% isoflurane using a rodent anesthesia machine. Twenty four hours prior to administration of elastase mice were administered saline (control), or 100,000 or 250,000 or 500,000 B regulatory cells isolated by CD5 expression from mice treated with 1 million MSC per mouse. Mice were sacrifice at the indicated timepoints and neutrophils were stained using hemoxylin staining and quantified by independent counting. Results are shown in FIG. 1.

1. A method of treating COPD by administration and/or generation of a therapeutic population of B cells.

2. The method of claim 1, wherein said COPD is associated with excessive production of elastase.

3. The method of claim 1, wherein said therapeutic B cells are B regulatory cells.

4. The method of claim 3, wherein said B regulatory cells express interleukin-10.

5. The method of claim 4, wherein said B regulatory cells express c-met.

6. The method of claim 4, wherein said B regulatory cells express thrombopoietin receptor.

7. The method of claim 4, wherein said B regulatory cells express c-kit.

8. The method of claim 4, wherein said B regulatory cells express CD5.

9. The method of claim 4, wherein said B regulatory cells express complement receptor 3.

10. The method of claim 4, wherein said B regulatory cells express interleukin-7 receptor.

11. The method of claim 4, wherein said B regulatory cells produce interleukin-35.

12. The method of claim 4, wherein said B regulatory cells are capable of inducing proliferation of pulmonary type 2 epithelial cells.

13. The method of claim 4, wherein said B regulatory cells, upon crosslinking of CD19 are capable of modifying nkt cells to produce a reduced amount of interleukin-17 as compared to control nkt cells.

14. The method of claim 4, wherein said B regulatory cells, upon crosslinking of CD19 are capable of modifying nkt cells to produce a reduced amount of TNF-alpha as compared to control nkt cells.

15. The method of claim 4, wherein said B regulatory cells, upon crosslinking of CD19 are capable of modifying nkt cells to produce a reduced amount of interferon gamma as compared to control nkt cells.

16. The method of claim 4, wherein said B regulatory cells, upon crosslinking of CD19 are capable of modifying nkt cells to produce a reduced amount of HMGB1 as compared to control nkt cells.

17. The method of claim 4, wherein said B regulatory cells, upon crosslinking of CD19 are capable of modifying nkt cells to produce an enhanced amount of interleukin-10 as compared to control nkt cells.

18. The method of claim 4, wherein said B regulatory cells, upon crosslinking of CD19 are capable of modifying nkt cells to produce an enhanced amount of soluble TNF-alpha receptor p55 as compared to control nkt cells.

19. The method of claim 4, wherein said B regulatory cells, upon crosslinking of CD19 are capable of modifying nkt cells to produce an enhanced amount of soluble TNF-alpha receptor p75 as compared to control nkt cells.

20. The method of claim 4, wherein said B regulatory cells, upon crosslinking of CD19 are capable of modifying nkt cells to produce an enhanced amount of interleukin-1 receptor antagonist as compared to control nkt cells.
